(19) United States
(12) Patent Application Publication
(10) Pub. No.: US 2012/0028930 A1
(43) Pub. Date: Feb. 2, 2012

(54) FLUPIRTINE SALTS AND POLYMORPHS

(75) Inventors: Isabel KALOFONOS, Cambridge, MA (US); William Martin Doyle, Cambridge, MA (US)

(73) Assignee: Bionevia Pharmaceuticals Inc., Cambridge, MA (US)

(21) Appl. No.: 13/035,663

(22) Filed: Feb. 25, 2011

Related U.S. Application Data
(60) Provisional application No. 61/308,557, filed on Feb. 26, 2010.

(51) Int. Cl. A61K 31/44; A61P 21/00; A61P 25/00; A61P 9/12; C07D 213/75; A61K 31/603

(52) U.S. Cl. 514/159; 546/308; 514/352

(57) ABSTRACT

The present invention includes salts and polymorphs of flupirtine including, but not limited to carboxylic acid salts and sulfonic acid salts and their polymorphs. The present invention also includes pharmaceutical compositions comprising the salts and polymorphs of flupirtine. The invention also relates to the therapeutic use of the flupirtine salts and polymorphs to treat nervous system disorders, pain disorders, musculoskeletal disorders, and other diseases and conditions.

### CROSS-REFERENCE TO RELATED APPLICATION

This application claims the benefit of U.S. Provisional Application No. 61/308,577, filed on Feb. 27, 2010 and entitled “FLUPIRTINE SALTS AND POLYMORPHS”, the contents of which are herein incorporated by reference in their entireties for all purposes.

### FIELD OF THE INVENTION

The invention relates to flupirtine salts and their crystalline forms, particularly to the carboxylic acid salts and sulfonic acid salts of flupirtine. The invention also relates to their therapeutic use to treat various diseases including nervous system disorders, pain disorders, pulmotary arterial hypertension, and musculoskeletal disorders, and to pharmaceutical compositions containing the salts and/or their polymorphs.

### BACKGROUND OF THE INVENTION

Flupirtine, 2-amino-3-carbethoxyamino-6-(p-fluorobenzylamino)pyridine, (shown below) is a known active pharmaceutical ingredient (API) having beneficial analgesic, muscle relaxant, neuroprotective, and other nervous system activities and is useful in treating pain, muscle contracture-related, and other nervous system conditions.

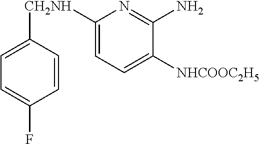

Flupirtine is an analgesic that has been used in Europe to treat the pain associated with surgery, cancer, trauma. It acts via the central nervous system through non-opiate pain pathways, possibly involving the thalamus or spinal pain pathways. In some, but not all, studies flupirtine has been found to be as effective as opiates in relieving pain. Moreover, flupirtine offers a clear advantage over opiates in that it is not addictive and there have been no reports of abuse. U.S. Pat. No. 6,610,324 describes flupirtine in the treatment of fibromyalgia and related conditions. The present invention is based upon, at least in part, the unexpected discovery that flupirtine is capable of completely eliminating the chronic and severe body pain present in some patients with fibromyalgia. Relief is possible even when high doses of opiates, non-steroidal anti-inflammatory drugs and Plaquenil fail to alleviate pain.

For example, flupirtine is therapeutically effective in the treatment of acute and chronic pain of various etiologies. Flupirtine also has positive indications for the treatment of neurodegenerative conditions. The preparation of flupirtine free base and a crystalline form of flupirtine hydrochloride salt are described in ZA Pat. No. 69 02364, German Pat. No. 1795858; U.S. Pat. No. 3,481,943; U.S. Pat. No. 4,785,110; von Bebenburg W et al., Chemiker-Zeitung 1979; 103:387; and von Bebenburg W et al., Chemiker-Zeitung 1981; 105:217-219. The preparation and characterization of various crystalline forms of the flupirtine maleate salt in German Pat. No. 31335191; U.S. Pat. No. 4,481,205; U.S. Pat. No. 5,959,115; WO2008/007117; Landgraf K F et al, European Journal of Pharmaceutics and Biopharmaceutics 46 (1998) 329-337; and Kuhnert-Brandstaetter M and Porsche U, Scientia Pharmaceutica 1990; 58; 55. The preparation of flupirtine gluconate is described in U.S. Pat. No. 4,673,666. Therapeutic activity of flupirtine maleate, which is the commercially available form, has been demonstrated in various conditions in the clinical literature, including but not limited to Bromm B et al., Postgrad Med J. 1987; 63 Suppl 3:109-12; Ceccarelli G et al., Postgrad Med J. 1987; 63 Suppl 3:105-8; Galasko C S et al., Curr Med Res Opin. 1985; 9:594-601; Göbel H et al., Schmerz. 1999 Oct. 15; 13(5):324-31; Goodchild C et al., Pain Medicine 2007; 8:612; Herrmann W M et al., Fortschr Med. 1993; 111:266-70; Hemnann W M et al., Postgrad Med J. 1987; 63 Suppl 3:87-103; Heusinger J H, Postgrad Med J. 1987; 63 Suppl 3:71-9; Lüben V et al., Fortschr Med. 1994; 112:282-6; Mastronardi P et al., J Int Med. Res. 1988; 16:338-48; McMahon F G et al., Postgrad Med J. 1987; 63 Suppl 3:81-5; Million R et al., Curr Med Res Opin. 1984; 9:204-12; Moore R A et al., Br Anaesth. 1983; 55:429-32; Müller-Schwefe G, Fortschr Med. Orig. 2003; 121:11-18; Müller-Schwefe G, MMW Fortschr Med. 2004; 146 Spec No 2:76; Müller-Schwefe G H et al., MMW Fortschr Med. 2007 25; 149:153-161; Otto M et al., Neurology. 2004; 62:714-8; Riethmüller-Winzen H, Postgrad Med J. 1987; 63 Suppl 3:61-5; Ringe J D et al., Arzneimittelforschung. 2003; 53:496-502; Salembier L et al., Acta Otolaryngol Suppl. 2006; 556:93-5; Scheef W et al., Arzneimittelforsdhung. 1985; 35:75-7; Scheef W, Postgrad Med J. 1987; 63 Suppl 3:67-70; Stoll A L, Psychosomatics. 2000; 41:371-2; Wörz R et al., Fortschr Med. 1996; 114:500-4; and Wörz R et al., Fortschr Med. 1995; 113:463-8; and in the patent prior art, including U.S. Pat. No. 4,668,684; U.S. Pat. No. 4,778,799; U.S. Pat. No. 5,162,346; U.S. Pat. No. 5,284,861; U.S. Pat. No. 5,521,178; U.S. Pat. No. 5,721,258; U.S. Pat. No. 6,034,111; U.S. Pat. No. 6,034,112; U.S. Pat. No. 6,124,326; U.S. Pat. No. 6,610,324; U.S. Pat. No. 6,821,995; U.S. Pat. No. 7,309,713; WO/2002/015907; WO/2005/000306; WO/2005/058319; WO/2006/079559; WO/2007/128462.

Although therapeutic efficacy is the primary concern for a therapeutic agent, like flupirtine, the salt or solid state form (i.e., the crystalline or amorphous form) of a drug candidate can be critical to its pharmacological properties and to its development as a viable API. For example, each salt or each crystalline form of a drug candidate can have different solid state (physical and chemical) properties. The differences in physical properties exhibited by a novel solid form of an API (such as a crystalline salts, or polymorph of the original compound), affect pharmaceutical parameters such as storage stability, compressibility and density (important in formulation and product manufacturing), and solubility and dissolution rates (important factors in determining bioavailability). Because these practical properties are influenced by the solid state form of the API, they can significantly impact the selection of a compound as an active pharmaceutical ingredient (API), the ultimate pharmaceutical dosage form, the optimization of manufacturing processes, and absorption in the body. Moreover, finding the most adequate form for further drug development can reduce the time and the cost of that development.

Obtaining pure crystalline forms, then, is extremely useful in drug development. It permits better characterization of the drug candidate's chemical and physical properties. Crystalline forms often have better chemical and physical properties than amorphous forms. The crystalline form may possess more favorable pharmacology than the amorphous form or be easier to process. It may also have better storage stability. The crystalline form can be a particular API itself, but more commonly are a salt of the API. In some instances. It is also possible to achieve desired properties of a particular API by forming a cocrystal of the API itself or of a salt of the API. Cocrystals are crystals that contain two or more non-identical molecules. Examples of cocrystals may be found in the Cambridge Structural Database. Examples of cocrystals may also be found at Etter, M. C., and Adsmond, D. A., J. Chem. Soc., Chem. Commun. 1990 589-591; Etter, M. C., MacDonald, J. C., and Bernstein, J., Acta Crystallogr., Sect. B, Struct. Sci. 1990 B46 256-262; and Etter, M. C., Urbańczyk-Lipkowska, Z., Zia-Ebrahimi, M., and Panunto, T. W., J. Am. Chem. Soc. 1990 112 8415-8426, which are incorporated herein by reference in their entireties. The following articles are also incorporated herein by reference in their entireties: Görbotz C. H., and Hersleth, H. P. Ada Cryst. 2000 B56 625-534; and Senthil Kumar, V. S., Nangia, A., Katz, A. K., and Carrell, H. L., Crystal Growth & Design, 2002 2 313-318.

One such physical property, which can affect processability, is the flowability of the solid, before and after milling. Flowability affects the ease with which the material is handled during processing into a pharmaceutical composition. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its dissolution rate in aqueous fluid. The rate of dissolution of an active ingredient in a patient's stomach fluid may have therapeutic consequences since it impacts the rate at which an orally administered active ingredient may reach the patient's bloodstream.

Another important solid state property of a pharmaceutical compound is its thermal behavior, including its melting point. The melting point of the solid form of a drug must be high enough to avoid melting or plastic deformation during standard processing operations, as well as concretion of the drug by plastic deformation on storage (Gould, P. L. Int. J. Pharmaceutics 1986 33 201-217). Normally a solid form should melt above about 100° C. to be considered optimum for development. For example, melting point categories used by one pharmaceutical company are, in order of preference, +(mp>120° C.), 0 (mp 80-120° C.), and −(mp<80° C.) (Balbach, S.; Korn, C. Int. J. Pharmaceutics 2004 275 1-12).

By crystallizing an API or a salt of an API, one creates a new solid state form of the API which has unique properties compared with existing non-crystalline forms, e.g., amorphous form, of the API or its salt. For example, a crystal may have different dissolution and solubility properties than the active agent itself or its salt. Crystals containing APIs can be used to deliver APIs therapeutically. New drug formulations comprising crystals of APIs or their salts may have superior properties over existing drug formulations.

A crystalline form of a compound, a crystalline salt of the compound containing the compound or its salt form generally possesses distinct crystallographic and spectroscopic properties when compared to other crystalline forms having the same chemical composition. Crystallographic and spectroscopic properties of the particular form are typically measured by X-ray powder diffraction (XRPD), single crystal X-ray crystallography, solid state NMR spectroscopy, e.g. ¹³C CP/MAS NMR, or Raman spectrometry, among other techniques. The particular crystalline form of a compound or of its salt often also exhibit distinct thermal behavior. Thermal behavior is measured in the laboratory by such techniques as capillary melting point, thermogravimetric analysis (TGA) and differential scanning calorimetry (DSC).

As mentioned above, German Pat. No. 1795858; U.S. Pat. No. 3,481,943; U.S. Pat. No. 4,785,110; von Bebenburg W et al., Chemiker-Zeitung 1979; 103:387; and von Bebenburg W et al., Chemiker-Zeitung 1981; 105:217-219 describe the synthesis of a family of compounds including flupirtine free base and a crystalline form of flupirtine hydrochloride. German Pat. No. 31335191, U.S. Pat. No. 4,481,205, U.S. Pat. No. 5,959,115, and WO2008/007117 describe the synthesis and basic activities of flupirtine maleate, which is the available form for therapeutic use. Hlavica P et al., Arzneimittelforschung 1985; 35:67-74 describes the pharmacokinetic parameters of flupirtine in man, including a time to maximum plasma concentration (tₘₐₓ) of 2 hours. As described in Macheras Petal., Pharmaceutical Research 2000; 17:108-112, the dissolution rate of a compound can have effects on and a drug with faster dissolution rate may also have a shorter tₘₐₓ. As described in Yüksel N, European Journal of Pharmaceutics and Biopharmaceutics 2003; 56:453-459, for drugs intended to be used as acute analgesics, a shorter tₘₐₓ is considered superior, since this results in a faster time to pain relief. Geisslinger G et al, Int J Clin Pharmacol Ther Toxicol 1989; 27:324-8 describe improving the tₘₐₓ and onset of action of ibuprofen free acid by administration of ibuprofen lysine salt, which has a faster dissolution rate than the free acid. Flupirtine maleate is reported to be insoluble in water (Sigma-Aldrich flupirtine maleate product information, 2008), and the dissolution rate of flupirtine maleate was measured at 0.088 [μg/mL]/min. A formulation of flupirtine with a higher dissolution rate than the flupirtine maleate salt may therefore have superior properties over existing drug formulations, particularly for use as an acute analgesic, for which a shorter tₘₐₓ is desirable.

### SUMMARY OF THE INVENTION

The present invention includes salts and polymorphs of flupirtine including, but not limited to, carboxylic acid salts and sulfonic acid salts and their polymorphs.

The present invention includes pharmaceutical compositions comprising the salts and polymorphs of flupirtine.

The present invention includes the therapeutic use of the salts and polymorphs of flupirtine.

### DETAILED DESCRIPTION OF THE INVENTION

The present invention provides various salts and polymorphs of flupirtine including, but not limited to, carboxylic acid salts and sulfonic acid salts and their polymorphs. Examples of flupirtine salts include, but are not limited to, flupirtine maleate, flupirtine decanoate, flupirtine succinate, flupirtine palmitate, flupirtine fumarate, flupirtine mesylate, flupirtine sulfonates, flupirtine sulfinates, flupirtine sulfoxide, flupirtine sulfone, flupirtine phosphate, flupirtine hydrobromide, flupirtine hydrochloride, flupirtine 1-hydroxy-2-naphthoate, flupirtine 2-oxoglutarate, flupirtine adipate, flupirtine alginate, flupirtine L-ascorbate, flupirtine L-aspartate, flupirtine benzoate, flupirtine bicarbonate, flupirtine bisulfate, flupirtine bitartrate, flupirtine camsylate, flupirtine carbonate, flupirtine citrate, flupirtine cyclamate, flupirtine edetate, flupirtine edisylate, flupirtine estolate, flupirtine galactarate, flupirtine gentisate, flupirtine gluceptate, flupirtine D-glucoheptonate, flupirtine D-glucoronate, flupirtine glutamate, flupirtine glutarate, flupirtine glycerophosphate, flupirtine glycolate, flupirtine glycollylarsanilate, flupirtine hexanoate, flupirtine hexylresorcinate, flupirtine hippurate, flupirtine hydroxynaphthoate, flupirtine isethionate, flupirtine isobutyrate, flupirtine lactate, flupirtine lactobionate, flupirtine laurate, flupirtine L-malate, flupirtine malonate, flupirtine mandelate, flupirtine methylbromide, flupirtine methylsulfate, flupirtine mucate, flupirtine napsylate, flupirtine nicotinate, flupirtine octanoate, flupirtine oleate, flupirtine orotate, flupirtine oxalate, flupirtine pamoate, flupirtine pantothenate, flupirtine polygalacturonate, flupirtine L-pyroglutamate, flupirtine salicylate, flupirtine stearate, flupirtine sulfate, flupirtine tannate, flupirtine tartrate, flupirtine teoclate, flupirtine thiocyanate, and flupirtine undecanoate.

In one embodiment, the present invention provides a crystalline salt of flupirtine, which is a carboxylic acid salt or a sulfonic acid salt. By “carboxylic acid”, it is meant an organic compound having one or more carboxyl groups, i.e., —C(═O)OH. The present carboxylic acid can be a mono-carboxylic acid, i.e., having one carboxyl group, or a bis-carboxylic acid, i.e., having two carboxyl groups. By “sulfonic acid”, it is meant an organic compound having one or more groups with the formula: —S(═O)₂OH.

In one embodiment, the carboxylic acid has a structural formula selected from the following:

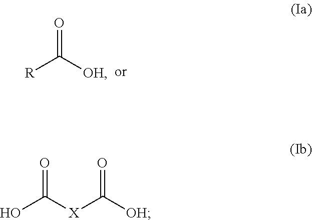

wherein R is a C₁ to C₂₀ alkyl, C₆ to C₁₀ aryl, or a C₂ to C₂₀ alkenyl; and X is a C₁ to C₆ alkylene or a C₂ to C₆ alkenylene. The alkyl, aryl, alkenyl, alkylene, alkenylene groups may be each independently substituted or unsubstituted.

In one embodiment, the sulfonic acid has a structural formula (II):

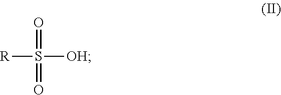

wherein R is a C₁ to C₂₀ alkyl, C₆ to C₁₀ aryl, or a C₂ to C₂₀ alkenyl. The alkyl, aryl, alkenyl, alkylene, alkenylene groups may be each independently substituted or unsubstituted.

In some embodiments, the present flupirtine salts are in their crystalline forms.

As referred to herein, the “aspect ratio” denotes the ratio of the second largest dimension of a crystal particle (i.e. its width) to the largest dimension of the crystal particle (i.e. its length). The aspect ratio may be determined from a representative number of crystals by visual observation under a light microscope.

As referred to herein, the term “substantially less acicular” denotes crystalline particles having an aspect ratio of 0.1 or greater. Preferably, the aspect ratio is between 0.2 and 1.0, preferably between 0.3 and 0.9, more preferably between 0.4 and 0.8, and most preferably between 0.6 and 0.7.

As referred to herein, the term “non-acicular crystals” denotes crystalline particles having an aspect ratio of 0.2 or greater. Preferably, the aspect ratio is between 0.2 and 1.0, preferably between 0.3 and 0.9, more preferably between 0.4 and 0.8, and most preferably between 0.6 and 0.7.

As referred to herein, the term “crystalline particles” preferably denotes crystalline flupirtine acid addition salts characterised as having a mean particle size of greater than 30 μm. Preferably, the mean particle size is greater than 50 μm, more preferably greater than 100 μm, even more preferably greater than 150 μm, and most preferably greater than 200 μm. Furthermore, for reasons of formulation efficiency, it is preferred that the mean particle size is less than 1000 μm, preferably less than 500 μm, even more preferably less than 300 μm, and most preferably less than 250 μm.

As defined herein, the term “particle size” refers to the size of the largest dimension of a crystal particle, and the mean particle size is determined from a representative number of crystals. The particle size may be determined by visual observation under a light microscope.

As referred to herein, the term “storage form” denotes a flupirtine salt to which the free flupirtine base is converted by acidification for the purpose of later conversion of said salt to another flupirtine salt. For example, according to the above definition, flupirtine acetate would be a storage form of flupirtine base if flupirtine acetate is produced and subsequently converted, via the base, to e.g. flupirtine gluconate.

The present invention relates to carboxylate acid addition salts of flupirtine that posses a non-acicular morphology in contrast to the previously known salts of flupirtine, i.e., these salts of the present invention crystallise as polygons, platelets, cubes or short columns. This is surprising since, as described in Chemiker Zeitung 105: 217-219, 1981, the linear structure of flupirtine free base and of flupirtine hydrochloride is thought to induce the formation of needle-like crystals.

In one embodiment, the present invention provides flupirtine carboxylate acid addition salts wherein the carboxylic acid has structural formula (Ia), wherein R represents hydrogen or a C₁-C₆ alkyl group excluding C₁-C₆ alkyl group that are of maleate and gluconate flupirtine salts.

Preferably, the salts have a non-acicular morphology.

Preferably, the C₁-C₆ alkyl group is a saturated C₁-C₆ alkyl group excluding C₁-C₆ alkyl group of gluconate flupirtine salt.

Suitable saturated C₁-C₆ alkyl group comprise methyl, ethyl, n-propyl, iso-propyl, n-butyl, sec-butyl, tert-butyl, pentyl and hexyl.

Preferably, the acid is formic, acetic or propionic acid, more preferably, acetic or propionic acid.

In the above acid addition salt, the mole ratio of the acid component to the flupirtine base component is 1:1 to 1:1.3, preferably 1:1.2, more preferably 1:1 to 1:1.1, and most preferably 1:1, respectively.

The present invention also relates to a population of the above flupirtine carboxylate acid addition salts characterised in that at least 50 mol-%, preferably at least 60 mol-%, more preferably at least 70 mol-%, even more preferably at least 80 mol-% and most preferably at least 90 mol-% of the crystalline particles have crystallized in the form of non-acicular crystals as defined above.

The non-acicular morphology of the salts of the invention can be exemplified by comparison of flupirtine acetate to flupirtine maleate.

The non-acicular morphology of the above flupirtine salts also results in a much higher bulk density. This is advantageous for packing and for controlled release formulations, since polygons and hexagons are more easily coated than acicular crystals and, thus, the formulation difficulties inherent to prior used acicular flupirtine salts are also overcome.

Furthermore, the markedly less acicular morphology of the crystals allows better flowability and dosing. Thus, the hexagonal or polygonal crystal shape of these flupirtine salts overcomes the processing difficulties inherent to acicular flupirtine salts such as flupirtine maleate.

In contrast to other acid addition salts, for example, maleate and gluconate, the carboxylic acid addition salts of flupirtine of the present invention, especially acetate and the propionate, show substantially improved stability towards oxidation and towards discoloration, when exposed to air for a storage period of 30 days at room temperature. Discoloration can take place after prolonged storage or when exposed to air. Discoloration is regarded as leading to an active pharmaceutical ingredient (API) of low pharmaceutical quality and can be avoided by using a protective atmosphere in the production process and using expensive packaging materials for the finished drug forms. The discoloration effect is known for flupirtine hydrochloride, flupirtine gluconate and both polymorphs of flupirtine maleate.

Further, the above salts may be more stable overall and more soluble in various solvents than the prior known flupirtine acid addition salts.

Accordingly, the alcohol component comprises at least 10 wt-%, preferably at least 20 wt-%, even more preferably at least 30 wt-%, and most preferably at least 40 wt-%, of the total weight of the solvents.

Preferred alcohols are pharmaceutically acceptable alcohols as are known in the art. Preferred examples include propylene glycol, ethanol, 2-(2-ethoxyethoxy)ethanol, benzyl alcohol, glycerol, glycofurol, polyethylene glycol 200, and the like. More preferred is ethanol.

The above acid addition salts may be prepared by a process comprising reacting free flupirtine base (2-amino-3-carbethoxyamino-6-(4-fluorobenzylamino-pyridine) and a carboxylic acid providing a reaction mixture from which the said acid addition salt precipitates.

The free flupirtine base can be prepared by known processes as are described, for example, in European patent no. EP 0 977 736, example 2.

In some embodiments, a solution comprising flupirtine base and a solvent is reacted with the acid thus providing the said reaction mixture, in others, flupirtine base, the acid and the solvent are combined providing the said reaction mixture.

In some preferred embodiments, the acid is dissolved in a solvent prior to the reaction with flupirtine base; in others the acid is reacted neat, i.e., without a solvent.

The solution can be prepared by combining flupirtine base and the solvent. Preferably, this combination is heated to aid in dissolution, if required. Preferably, heating is to a temperature of about 30° C. to about 80° C. Preferably, to avoid oxidation, a protective atmosphere like nitrogen or argon may be used in this reaction.

Examples for suitable solvents include but are not limited to ethers, alcohols, ketones, esters, halogenated solvents, nitriles, water or mixtures thereof.

Preferably, the ethers are C₃-C₇ ethers, the alcohols are C₁-C₅ alcohols, the ketones are C₃-C₅ ketones, the esters are C₂-C₆ esters, the halogenated solvents are C₁-C₆ halogenated solvents, the nitriles are C₂-C₃ nitriles. More preferably, the C₁-C₆ halogenated solvents are C₁-C₃ chlorinated solvents and the C₂-C₆ esters are C₂-C₆ acetates.

Preferably, the C₃-C₇ ethers are diethyl ether, diisopropyl ether, t-butyl methyl ether, or mixtures thereof. Preferably, the C₁-C₅ alcohols are methanol, ethanol, isopropanol, n-propanol, n-, iso-, or tert-butanol, or mixtures thereof. Preferably, the C₃-C₅ ketones are acetone, methyl ethyl ketone, diethyl ketone, or mixtures thereof. Preferably, the C₁-C₆ halogenated solvents are dichloromethane, chloroform or chlorobenzene, or mixtures thereof; the C₂-C₃ nitriles are acetonitrile, propionitrile, or mixtures thereof; the C₂-C₆ acetates are methyl-, ethyl-, n-propyl-, i-propyl-, butyl-acetate or mixtures thereof. Most preferably, the C₁-C₃ alcohols are methanol, ethanol or iso-propanol or mixtures thereof. Most preferably, the solvent is a C₁-C₅ alcohol, more preferably ethanol or iso-propanol.

It is further preferred that the flupirtine base is reacted with about 1 to about 1.5 mole equivalents, preferably about 1 to about 1.2 mole equivalents, and most preferably about 1 to about 1.1 mole equivalents, of the corresponding carboxylic acid per mole equivalent of flupirtine base. Preferably, the carboxylic acid is acetic acid or propionic acid.

Preferably, the said reaction mixture is preferably a solution from which the acid addition salt precipitates.

Precipitation occurs either on its own motion or is induced by reducing the solvent volume and/or the temperature and/or by adding an anti solvent and/or by adding seeding crystals.

Typically, the anti-solvent is such that is less polar than the reaction solvent and thus when added leads to the formation of a precipitate of the said salt. Examples for such solvent can be anyone of the above and also toluene, benzene, hexane, cyclohexane and pentane.

Preferably, the temperature is reduced to about 0° C. to about −20° C.

The precipitated salt is collected by filtration and drying, optionally followed by further purification steps. Preferably, drying is performed under reduced pressure for about 12 hours.

According to this procedure flupirtine salts of high purity are obtained. Preferably the purity is of more than about 95 area percent as measured by HPLC. Thus, these salts are especially suitable for the purification of flupirtine base since they crystallize in polygonal dense shapes and are less likely to contain enclosures of solvents and/or impurities.

The structure and composition of the obtained salts prepared can be confirmed by analytical methods such as NMR spectrometry, IR spectrometry, elemental analysis, DSC analysis, and the determination of the melting point.

The salts prepared by the method described above can be further purified by recrystallization from appropriate solvents, if necessary. Examples for such solvents include iso-propanol, acetone, methylene chloride and chloroform.

Furthermore, the salts of the invention can further be purified by conversion to the flupirtine free base, followed by extraction or crystallization and renewed acidification.

In the above process, when the acid is acetic acid and the solvent is at least one of ethers, alcohols, ketones, esters, halogenated solvents, nitriles, water or mixtures thereof, a crystalline form of flupirtine acetate is obtained. The preferred list of solvents is mentioned before.

In one embodiment, the present invention provides a crystalline form of flupirtine acetate characterized by a powder XRD pattern with peaks showing particular degrees two theta.

The above crystalline form of flupirtine acetate can be further characterized by a DSC peak, a DSC pattern, and a FT-IR spectrum with particular peaks, and an IR pattern; and a combination thereof.

In the above process, when the acid is propionic acid and the solvent is at least one of ethers, alcohols, ketones, esters, halogenated solvents, nitriles, water or mixtures thereof, a crystalline form of flupirtine propionate is obtained. The preferred list of solvents is mentioned before.

In one embodiment, the present invention provides a crystalline form of flupirtine propionate is characterized by a powder XRD pattern with peaks showing particular degrees two theta.

The above crystalline form of flupirtine propionate can be further characterized by a DSC peak, a DSC pattern, and a FT-LR spectrum with particular peaks, and an IR pattern; and a combination thereof.

In one embodiment, the present invention provides flupirtine carboxylate acid addition salts wherein the carboxylic acid has structural formula (Ib), wherein X is a C₂ to C₆ alkenylene. Examples of the carboxylic acid include, but are not limited to fumaric acid, maleic acid, and etc.

In one embodiment, the present invention provides one or more crystalline forms of flupirtine maleate is characterized by a powder XRD pattern with peaks showing particular degrees two theta.

The above crystalline forms of flupirtine maleate can be further characterized by a DSC peak, a DSC pattern, and a FT-IR spectrum with particular peaks, and an IR pattern; and a combination thereof.

The present polymorphic forms of flupirtine maleate may exhibit beneficial properties and, in particular, provide advantages over commercially available flupirtine maleate. The advantages can be increased physical stability, improved dissolution, improved morphology, improved properties when formulated, and improved properties during storage.

The present invention includes sulfonic acid addition salts wherein the sulfonic acid has structural formula (II), wherein R represents a substituted or un-substituted C₁-C₁₂-alkyl or C₆-C₁₀-aryl group.

Suitable C₁-C₁₂-alkyl groups comprise methyl, ethyl, n-propyl, isopropyl, n-butyl, sec-butyl, tert-butyl, pentyl, hexyl, cyclohexyl, heptyl, octyl, nonyl, decyl and dodecyl.

Suitable C₆-C₁₀-aryl groups comprise phenyl and naphthyl.

The or C₁-C₁₂-aryl or C₆-C₁₀-aryl groups may be substituted. Suitable substituents include and are not limited to hydroxyl, C₁-C₄-alkoxy, phenoxy, halogen, COOH, C₁-C₄-alkyl, and phenyl.

Preferred substituents include hydroxyl, methoxy, ethoxy, phenoxy, fluoro, chloro, bromo, iodo, methyl, ethyl, n-propyl, isopropyl, n-butyl, sec-butyl, tert-butyl and phenyl as well as further sulfonic acid groups.

Preferred examples of acid components in above formula (2) comprise methane sulfonic acid, ethane sulfonic acid, n-propane sulfonic acid, n-butane sulfonic acid, dodecane sulfonic acid, phenylethane sulfonic acid, hydroxyethane sulfonic acid, methoxyethane sulfonic acid, benzene sulfonic acid, halogenbenzene sulfonic acid, methoxybenzene sulfonic acid, toluene sulfonic acid, 5-salicylsulfonic acid, naphthalene sulfonic acid, and naphthalene disulfonic acid.

Preferred examples of halogenbenzene sulfonic acid comprise 4-fluorobenzene sulfonic acid 4-chlorobenzene sulfonic acid and 4-bromobenzene sulfonic acid.

Preferred examples of naphthaline sulfonic acid include 1-naphthaline sulfonic acid and 2-naphthaline sulfonic acid.

Preferably, the acid component in the above-mentioned sulfonic acid salt is methane sulfonic acid or benzene sulfonic acid.

The mole ratio of the sulfonic acid component to the flupirtine base component is 1:1 to 1:1.5, preferably 1:1.2, more preferably 1:1 to 1:1.1, and most preferably 1:1, respectively.

Furthermore, the present invention also relates to a population of crystalline flupirtine sulfonic acid addition salts characterised in that at least 50 mol-%, preferably at least 60 mol-%, more preferably at least 70 mol-%, even more preferably at least 80 mol-% and most preferably at least 90 mol-% of the crystalline particles have a non-acicular morphology.

For example, flupirtine mesylate crystallizes in the form of short columns. The substantially less acicular morphology of the salts of the invention can be exemplified by comparison of flupirtine mesylate to flupirtine maleate.

Furthermore, the markedly less acicular morphology of the crystals may allow better flowability and dosing. Thus, the improved crystal shape of the flupirtine salts can overcome the processing difficulties inherent to acicular flupirtine salts such as flupirtine maleate.

The less acicular morphology of the present flupirtine salts can also result in a much higher bulk density, which is advantageous for formulations.

In contrast to other acid addition salts, for example, maleate and gluconate or inorganic salts, the sulfonate acid addition salts of flupirtine of the present invention, especially mesylate and the besilate, show substantially improved stability towards oxidation and towards discolouration, when exposed to air for a storage period of 30 days at room temperature. Discolouration can occur after prolonged storage or when exposed to air. This effect can be simulated using stress conditions: Elevated temperature and/or moisture. Discolouration is regarded as leading to an active pharmaceutical ingredient (API) of low pharmaceutical quality and can be avoided by using a protective atmosphere in the production process and using expensive packaging materials for the finished drug forms. The discolouration effect is known for flupirtine hydrochloride, flupirtine gluconate and both polymorphs of flupirtine maleate.

Furthermore, under dry conditions, in the absence of oxygen, the sulfonic acid addition salts of the present invention are also characterized by improved thermal stability as is evident from stress tests under nitrogen at elevated temperatures (e.g. 70° C.).

Consequently, the present salts may have superior storage stability than prior known flupirtine salts.

In addition, the present salts may not form solvates with a variety of solvents such as alcohols, water or methylene chloride. This is a significant advantage, because it is difficult to remove solvents from an API when preparing a formulation. The level of difficulty depends on the particle morphology and the temperature at which desolvatation occurs. For the flupirtine maleate salts, the temperature of desolvatation is from about 80° C. to about 100° C., which is relatively high. The amount of impurities in the salt could increase if heated. The absence of solvates can be determined by DSC analysis.

In another aspect of the invention, the salts of flupirtine with organosulfonic acids having 6 or less than 6 carbon atoms show better solubility in aqueous media compared to the currently available maleate form by factor 2 to 3.25.

The sulfonic acid addition salts may be prepared by a process comprising reacting flupirtine base (2-amino-3-carbethoxyamino-6-p-fluorobenzylamino-pyridine) an acid having the following formula

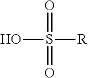

providing a reaction mixture from which the said acid addition salt precipitates, wherein R represents a substituted or unsubstituted or C₁-C₁₂-alkyl or C₆-C₁₀-aryl group.

In some embodiments, the addition salts are prepared by reacting flupirtine and an acid in a solvent providing a reaction mixture from which the said acid addition salt precipitates. In preferred embodiments, a solution of flupirtine in the solvent is reacted with the acid.

Preferably, the reaction mixture is heated to aid the salt formation, if required. Preferably, heating is to a temperature of about 30° C. to about 80° C. Preferably, to avoid oxidation, a protective atmosphere like nitrogen or argon may be used in this reaction.

Examples for suitable solvents include but not limited to ethers, alcohols, ketones, esters, halogenated solvents, nitriles, water, or mixtures thereof.

Preferably, the ethers are C3-C7 ethers, the alcohols are C1-C5 alcohols, the ketones are C3-C5 ketones, the esters are C2-C6 esters, the halogenated solvents are C1-C6 halogenated solvents and the nitriles are C2-C3 nitriles. More preferably, the C1-C6 halogenated solvents are C1-C6 chlorinated solvents and the C2-C6 esters are C2-C6 acetates.

Preferably, the C3-C7 ethers are diethyl ether, diisopropyl ether, or t-butyl methyl ether, or mixtures thereof. Preferably, the C1-C5 alcohols are methanol, ethanol, isopropanol, n-propanol, n-, iso-, or tert-butanol, or mixtures thereof. Preferably, the C3-C5 ketones are acetone, methyl ethyl ketone, diethyl ketone or mixtures thereof. Preferably, the C2-C6 esters are methyl-, ethyl-, n-propyl-, i-propyl-, butyl-acetate, or mixtures thereof. Preferably, the C1-C6 halogenated solvents are dichloromethane (DCM), chloroform, cholorobenzene or mixtures thereof. Preferably, the C2-C3 nitriles are acetonitrile, priopionitrile or mixtures thereof. Most preferably, the C1-C5 alcohols are ethanol or isopropanol. Most preferably, the solvent is a C1-C5 alcohol, more preferably ethanol or isopropanol.

It is further preferred that the solution of flupirtine is reacted with about 1 to about 1.3 mole equivalents, preferably about 1 to about 1.2 mole equivalents, most preferably about 1 to about 1.1 mole equivalents, of the corresponding sulfonic acid per mole equivalent of flupirtine base. Preferably, the sulfonic acid is methane sulfonic acid or benzene sulfonic acid.

Preferably, the said reaction mixture is preferably a solution from which the acid addition salt precipitates.

Precipitation occurs either on its own motion or is induced by reducing the solvent volume and/or the temperature and/or by adding an anti-solvent and/or by adding seeding crystals.

Typically, the anti-solvent is such that is less polar than the reaction solvent and thus when added to the solution leads to the formation of a precipitate of the said salt. Examples for such solvent can be anyone of the above solvents as well as toluene, benzene, hexane, cyclohexane and pentane.

The precipitated salt is collected by filtration and drying, optionally followed by further purification steps. Preferably, drying is performed under reduced pressure for about 12 hours.

According to this procedure flupirtine sulfonic acid salts of high purity are obtainable. Preferably, the purity is of more than about 95 Area percent as measured by HPLC. The structure and composition of the obtained salts prepared can be confirmed by the usual analytical methods such as NMR spectrometry, IR spectrometry, elemental analysis, DSC analysis, and the determination of the melting point.

The salts prepared by the method described above can be further purified by recrystallization from appropriate solvents, if necessary. Examples of such solvents include iso-propanol, acetone, methylene chloride and chloroform.

Furthermore, the salts of the invention can further be purified by conversion to the flupirtine free base, followed by extraction or crystallization and renewed acidification.

In the above process for preparing the sulfonic acid addition salt, the sulfonic acid is selected from methane sulfonic acid, ethane sulfonic acid, n-propane sulfonic acid, n-butane sulfonic acid, dodecane sulfonic acid, phenylethane sulfonic acid, hydroxyethane sulfonic acid, methoxyethane sulfonic acid, benzene sulfonic acid, halogenbenzene sulfonic acid, methoxybenzene sulfonic acid, toluene sulfonic acid, 5-salicylsulfonic acid, naphthalene sulfonic acid, and naphthalene disulfonic acid.

Preferred examples of halogenbenzene sulfonic acid comprise 4-fluorobenzene sulfonic acid 4-chlorobenzene sulfonic acid and 4-bromobenzene sulfonic acid.

Preferred examples of naphthaline sulfonic acid include 1-naphthaline sulfonic acid and 2-naphthaline sulfonic acid.

Preferably, the acid component is methane sulfonic acid or benzene sulfonic acid.

When the acid is methane sulfonic acid and the solvent is, preferably, selected from the list consisting of ethers, alcohols, ketones, esters, halogenated solvents, water or mixtures thereof, a crystalline form of flupirtine mesylate is obtained. The preferred list of solvents is mentioned before.

In one embodiment, the present invention provides a crystalline form of flupirtine mesylate characterized by a powder XRD pattern with peaks showing particular degrees two theta.

The above crystalline form of flupirtine mesylate can be further characterized by a DSC peak, a DSC pattern, a FT-IR spectrum with particular peaks; and a combination thereof.

Also, when the acid is benzene sulfonic acid and the solvent is, preferably, selected from the list consisting of ethers, alcohols, ketones, esters, halogenated solvents, water or mixtures thereof, a crystalline form of flupirtine besilate is obtained. The preferred list of solvents is mentioned before.

In one embodiment, the present invention provides a crystalline form of flupirtine besilate, characterized by a powder XRD pattern with peaks showing particular degrees two theta.

The above crystalline form of flupirtine besilate can be further characterized by a DSC peak, a DSC pattern, a FT-IR spectrum with particular peaks; and a combination thereof.

The present invention also provides pharmaceutical compositions comprising flupirtine salts and polymorphs thereof and the therapeutic use of the salts and polymorphs. In one embodiment, the present salts and polymorphs are used for neuroprotection and/or treatment of pain. In some specific embodiments, pain includes acute pain, neuropathic pain, fibromyalgia, and combinations thereof. In another specific embodiment, the present salts and polymorphs are used for treating huntington disease.

The flupirtine salts and polymorphs of the present invention possess the same pharmacological activity as flupirtine free base and its salts, such as flupirtine maleate, and are useful for treating nervous system disorders, pain disorders, and musculoskeletal conditions such as those discussed above, especially acute and chronic pain of various etiologies, including back pain, neck pain, pain resulting from traumatic injury, post-operative pain, post-dental procedure pain, dysmenorrhea, osteoarthritis, visceral pain, cancer pain, rheumatoid arthritis, psoriatic arthritis, gout, tendonitis pain, bursitis pain, musculoskeletal pain, sports injury-related pain, sprains, strains, pain of osteoporosis, ankylosing spondylitis, headache of various etiologies including but not limited to migraine and tension headache, temporomandibular joint pain, fibromyalgia, myofascial pain syndrome, pain of irritable bowel syndrome, interstitial cystitis, and idiopathic chronic pain.

The flupirtine salts and polymorphs of the present invention are also useful for treating acute and chronic neuropathic pain, and pain associated with nervous system disorders, including but not limited to, painful diabetic neuropathy, postherpetic neuralgia, trigeminal neuralgia, complex regional pain syndrome I, complex regional pain syndrome II, ischemic neuropathy, phantom limb pain, chemotherapy-induced neuropathy, HIV-related neuropathy, AIDS-related neuropathy, neuropathic back pain, neuropathic neck pain, carpal tunnel syndrome, other forms of nerve entrapment or nerve compression pain, brachial plexus lesions, other peripheral nerve lesions, neuropathic cancer pain, vulvodynia, central neuropathic pain, pain due to multiple sclerosis, post-stroke pain, Parkinson's Disease related central pain, postoperative chronic pain, Guillain-Barre syndrome (GBS), Charcot-Marie-Tooth (CMT) disease, idiopathic peripheral neuropathy, alcoholic neuropathy, other types of neuropathic pain, and other nervous system disorders that have pain as an attendant sign and/or symptom.

The flupirtine salts and polymorphs of the present invention exert a muscle relaxant effect, and is also useful for treating acute and chronic conditions of pathological muscle contracture, including but not limited to the discomfort, muscle spasm, stiffness, or myotonic conditions associated with painful musculoskeletal conditions, such as back pain, neck pain, neck-shoulder-arm syndrome, scapulohumeral periarthritis, cervical spondylosis, and other musculoskeletal conditions; spasticity or spastic paralysis of neurological origin due to multiple sclerosis, spinal cord injury, traumatic brain injury, cerebral palsy, stroke or cerebrovascular disorder, spastic spinal paralysis, sequelae of surgical trauma (including cerebrospinal tumor), amyotrophic lateral sclerosis, spinocerebellar degeneration, spinal vascular disorders, subacute myelo-optico neuropathy (SMON) and other encephalomyelopathies, and other neurological conditions; primary dystonia; secondary dystonia; and muscle cramps.

The flupirtine salts and polymorphs of the present invention have nervous system activity and neuroprotective effects, and are also useful for treating a variety of nervous system conditions including, but not limited to epilepsy, Creutzfeldt-Jakob Disease, Alzheimer's Disease, Parkinson's Disease, Huntington's Disease, Batten Disease, cerebral ischemia, schizophrenia, psychosis, mood disorders including bipolar disorder, major depressive disorder, dysthymia, anxiety disorders, overactive bladder, urinary incontinence, urinary flow problems as a result of prostate hyperplasia, irritable bowel syndrome, and tinnitus

The flupirtine salts and polymorphs of the present invention are also useful for treating diabetes mellitus and neurodegenerative diseases of the nervous and visual systems resulting as a complication of diabetes, including but not limited to diabetic neuropathy, diabetic retinopathy, diabetic maculopathy, glaucoma, diabetic gastroparesis, cataracts, and foot ulcers; for preventing and treating diseases associated with an impairment of the hematopoietic cell system, including but not limited to HIV and AIDS; for preventing and treating disorders which are associated with an unphysiologically high cell death rate, including but not limited to organ disorders resulting from myocardial infarct, cardiogenic shock, kidney shock, lung shock, and to other disorders associated with a high cell death rate including but not limited to senile macular degeneration and traumas resulting from mechanical, thermal, radiation, or other toxic influences.

The flupirtine salts and polymorphs of the present invention are also useful for treating hypertensions including pulmonary hypertension, such as pulmonary arterial hypertension and pulmonary venous hypertension.

As discussed, the invention relates to pharmaceutical compositions comprising a therapeutically effective amount of the flupirtine salts and polymorphs of the present invention and a pharmaceutically acceptable carrier (also known as a pharmaceutically acceptable excipient). The flupirtine salts and polymorphs of the present invention have the same pharmaceutical activity as previously reported for flupirtine and its salts, such as flupirtine maleate. Pharmaceutical compositions for the treatment of those conditions or disorders contain a therapeutically effective amount of the flupirtine salts and polymorphs of the present invention, as appropriate, for treatment of a patient with the particular condition or disorder. A “therapeutically effective amount” of the flupirtine salts and polymorphs, according to the invention (discussed here concerning the pharmaceutical compositions), refers to an amount of a therapeutic agent to treat or prevent a condition treatable by administration of a composition of the invention. That amount is the amount sufficient to exhibit a detectable therapeutic or preventative or ameliorative effect. The effect may include, for example, treatment or prevention of the conditions listed herein. The actual amount required for treatment of any particular patient will depend upon a variety of factors including the disorder being treated and its severity; the specific pharmaceutical composition employed; the age, body weight, general health, sex and diet of the patient; the mode of administration; the time of administration; the route of administration; and the rate of excretion of flupirtine; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman and L. Limbird, eds., McGraw-Hill Press, 155-173, 2001, which is incorporated herein by reference.

A pharmaceutical composition of the invention may be any pharmaceutical form which contains the flupirtine salts and polymorphs of the present invention. Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition of the invention, that is one having the flupirtine salts and polymorphs of the present invention, a carrier should be chosen that maintains its crystalline form. In other words, the carrier should not substantially alter the crystalline form of the flupirtine salts and polymorphs of the present invention. Nor should the carrier be otherwise incompatible with flupirtine itself or the flupirtine salts and polymorphs of the present invention, such as by producing any undesirable biological effect or otherwise interacting in a deleterious manner with any other component(s) of the pharmaceutical composition.

The pharmaceutical compositions of the invention are preferably formulated in unit dosage form for ease of administration and uniformity of dosage. A “unit dosage form” refers to a physically discrete unit of therapeutic agent appropriate for the patient to be treated. It will be understood, however, that the total daily dosage of the flupirtine salts and polymorphs of the present invention and its pharmaceutical compositions according to the invention will be decided by the attending physician within the scope of sound medical judgment.

Because the flupirtine salts and polymorphs of the present invention exist in crystalline forms, solid dosage forms are a preferred form for the pharmaceutical composition of the invention. Solid dosage forms for oral administration include capsules, tablets, pills, powders, and granules. Tablets are particularly preferred. The active ingredient may be contained in a solid dosage form formulation that provides quick release, sustained release or delayed release after administration to the patient. In such solid dosage forms, the active compound is mixed with at least one inert, pharmaceutically acceptable carrier such as sodium citrate or dicalcium phosphate. The solid dosage form may also include one or more of: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar, calcium carbonate, potato or tapioca starch, alginic acid, certain silicates, and sodium carbonate; e) dissolution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, and sodium lauryl sulfate. The solid dosage forms may also comprise buffering agents. They may optionally contain opacifying agents and can also be of a composition that they release the active ingredient(s) only, or preferentially, in a certain part of the intestinal tract, optionally, in a delayed manner. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Solid dosage forms of pharmaceutical compositions of the invention can also be prepared with coatings and shells such as enteric coatings and other coatings well known in the pharmaceutical formulating art.

The flupirtine salts and polymorphs of the present invention can be in a solid micro-encapsulated form with one or more carriers as discussed above. Microencapsulated forms may also be used in soft and hard-filled gelatin capsules with carriers such as lactose or milk sugar as well as high molecular weight polyethylene glycols and the like.

The flupirtine salts and polymorphs may also be used in the preparation of non-solid formulations, e.g., injectables and patches, of flupirtine. Such non-solid formulations are known in the art. In a non-solid formulation, the crystalline form is, generally speaking, not maintained. For example, the crystalline form may be dissolved in a liquid carrier. In this case, the crystalline forms of the invention represent intermediate forms of flupirtine used in the preparation of the non-solid formulation. The crystalline forms of the invention provide advantages of handling stability and purity to the process of making such formulations.

The flupirtine salts and polymorphs may also be formulated into a short acting injectable composition or dosage form or a long acting injectable composition or dosage form. Long acting injectables are important in the treatment of schizophrenia given low compliance of many patients.

Furthermore, the flupirtine salts and polymorphs may also be formulated into a sustained or delayed release dosage form which can provide therapeutically effective average steady-state plasma of flupirtine concentration when administered once or twice per day.

In the pharmaceutical compositions of the present invention for oral, sublingual, subcutaneous, intramuscular, intravenous, topical, intratracheal, intranasal, transdermal or rectal administration, a polymorphic form of flupirtine salts according to the present invention is administered to animals and humans in unit forms of administration, mixed with conventional pharmaceutical carriers, for the prophylaxis or treatment of the above disorders or diseases. The appropriate unit forms of administration include forms for oral administration, such as tablets, gelatin capsules, powders, granules and solutions or suspensions to be taken orally, forms for sublingual, buccal, intratracheal or intranasal administration, forms for subcutaneous, intramuscular or intravenous administration and forms for rectal administration. For topical application, a polymorphic form of flupirtine salts according to the present invention can be used in creams, ointments or lotions. Oral administration is preferred.

To achieve the desired prophylactic or therapeutic effect, the dose of a polymorphic form of flupirtine salts according to the present invention can vary between 0.01 and 50 mg per kg of body weight per day. Each unit dose can contain from 0.1 to 1000 mg, preferably 1 to 500 mg, of a polymorphic form of flupirtine salts according to the present invention in combination with a pharmaceutical carrier. This unit dose can be administered 1 to 5 times a day so as to administer a daily dosage of 0.5 to 5000 mg, preferably 1 to 2500 mg.

When a solid composition in the form of tablets is prepared, a polymorphic form of flupirtine salts according to the present invention is mixed with a pharmaceutical vehicle such as gelatin, starch, lactose, magnesium stearate, talc, gum arabic or the like. The tablets can be coated with sucrose, a cellulose derivative or other appropriate substances, or else they can be treated so as to have a prolonged or delayed activity and so as to release a predetermined amount of active principle continuously.

A preparation in the form of gelatin capsules can be obtained by mixing a polymorphic form of flupirtine salts according to the present invention with a diluent and pouring the resulting mixture into soft or hard gelatin capsules.

A preparation in the form of a syrup or elixir or for administration in the form of drops can contain a polymorphic form of flupirtine salts according to the present invention typically in conjunction with a sweetener, which is preferably calorie-free, optionally antiseptics such as methylparaben and propylparaben, as well as a flavouring agent and an appropriate colour.

Water-dispersible granules or powders can contain a polymorphic form of flupirtine salts according to the present invention mixed with dispersants or wetting agents, or suspending agents such as polyvinylpyrrolidone, as well as with sweeteners or taste correctors.

Rectal administration is effected using suppositories prepared with binders which melt at the rectal temperature, for example polyethylene glycols.

Parenteral administration is effected using aqueous suspensions, isotonic saline solutions or sterile and injectable solutions which contain pharmacologically compatible dispersants and/or wetting agents, for example propylene glycol or butylene glycol.

A polymorphic form of flupirtine salts according to the present invention can also be formulated as microcapsules, with one or more carriers or additives if appropriate.

The invention also relates to the treatment of nervous system disorders, pain disorders, and musculoskeletal disorders such as those discussed above. The invention provides a method for treating of nervous system disorders, pain disorders, and musculoskeletal disorders by administering to mammals the flupirtine salts and polymorphs of the present invention, or a pharmaceutical composition containing it, in an amount sufficient to treat or prevent a condition treatable by administration of a composition of the invention. That amount is the amount sufficient to exhibit a detectable therapeutic or preventative or ameliorative effect. The effect may include, for example, treatment or prevention of the conditions listed herein. The crystalline salts and pharmaceutical compositions containing it, according to the invention, may be administered using any amount, any form of pharmaceutical composition and any route of administration effective for the treatment. After formulation with an appropriate pharmaceutically acceptable carrier in a desired dosage, as known by those of skill in the art, the pharmaceutical compositions of this invention can be administered to humans and other animals orally, rectally, or topically (as by powders or other solid form-based topical formulations). In certain embodiments, the flupirtine salts and polymorphs of the present invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. As discussed above, the amount required for treatment of a particular patient will depend upon a variety of factors including the disorder being treated and its severity; the specific pharmaceutical composition employed; the age, body weight, general health, sex and diet of the patient; the mode of administration; the time of administration; the route of administration; and the rate of excretion of flupirtine; the duration of the treatment; any drugs used in combination or coincidental with the specific compound employed; and other such factors well known in the medical arts. And, as also discussed, the pharmaceutical composition of the flupirtine salts and polymorphs may be administered as a unit dosage form.

In one embodiment, the present invention provides pharmaceutical compositions comprising at least one of the above flupirtine acid addition salts and at least one pharmaceutically acceptable excipient. Optionally, prior known flupirtine salts such as flupirtine maleate and gluconate can also be used in addition to the compounds of the invention.

The pharmaceutical composition of the invention may also comprise one or more auxiliary excipients such as for example carriers, diluents, binders, lubricants, surfactants, disintegrants, plasticisers, anti-tack agents, opacifying agents, pigments, anti-oxidants and the like. As will be appreciated by those skilled in the art, the exact choice of excipient and relative amount will depend on the type of pharmaceutical composition, the API, dosage and other factors.

In another embodiment, the present invention also encompasses a pharmaceutical composition comprising at least one of the above described flupirtine acid addition salts prepared according to the processes of the present invention, and at least one pharmaceutically acceptable excipient.

In another embodiment, the invention encompasses a process for preparing a pharmaceutical composition comprising at least one of the above-described acid addition salts, and at least one pharmaceutically acceptable excipient.

In another embodiment, the invention encompasses the use of at least one of the above described flupirtine acid addition salts for the manufacture of a medicament for the treatment and prevention of acute and chronic pain, pain associated with degenerative and inflammatory joint disease, muscular tension, muscle spasm, muscle stiffness, neurodegenerative disorders, dementia, encephalopathy, diseases of the eye, myocardial ischemia and infarction, cerebral ischemia and infarction, shock, tinnitus and hepatitis.

In another embodiment, the invention encompasses the use of at least one of the above described flupirtine acid addition salts for the manufacture of a pharmaceutical composition.

In yet another embodiment, the invention encompasses a method of treating and preventing of acute and chronic pain, pain associated with degenerative and inflammatory joint disease, muscular tension, muscle spasm, muscle stiffness, neurodegenerative disorders, dementia, encephalopathy, diseases of the eye, myocardial ischemia and infarction, cerebral ischemia and infarction, shock, tinnitus and hepatitis, comprising administering a pharmaceutical composition comprising at least one of the above described flupirtine acid addition salts to a patient in need thereof.

As a result of these many and varied activities, the polymorphic forms of flupirtine maleate, as provided by the present invention, have a unique spectrum of pharmacological activity. They have utility in the treatment and prevention of acute and chronic pain including neuropathic pain, nerve pain, cancer pain, vasomotor and migraine headaches, post-operative pain, post-traumatic pain, burn pain, erosion pain, dysmenorrhoea, dental pain and the pain associated with degenerative and inflammatory joint disease.

They also have utility in the treatment and prevention of muscular tension, muscle spasm and muscle stiffness. They are particularly useful in the treatment of back pain.

Additionally, the polymorphic forms of flupirtine maleate, as provided by the present invention, exert potent cyto- and neuroprotective effects and have utility in the treatment and prevention of neurodegenerative disorders such as Parkinson's disease, dementia including Alzheimer's disease, Huntington's chorea, multiple sclerosis, amyotrophic lateral sclerosis, encephalopathy including AIDS related encephalopathy, Creutzfeldt-Jakob disease including classical and new-variant types and Batten disease.

They also have utility in the treatment and prevention of diseases of the eye such as maculopathy including senile macular degeneration, diabetic retinopathy, glaucoma and retinitis pigmentosa.

They also have utility in the treatment and prevention of myocardial ischaemia and infarction, cerebral ischaemia and infarction, shock, tinnitus and hepatitis.

The present invention further provides, therefore, pharmaceutical compositions comprising a therapeutically effective dose of a polymorphic form of flupirtine maleate according to the invention, together with a pharmaceutically acceptable carrier, diluent or excipient therefor. Excipients are chosen according to the pharmaceutical form and the desired mode of administration.

As used herein, the term “therapeutically effective amount” means an amount of a polymorphic form of flupirtine maleate according to the invention, which is capable of preventing, ameliorating or eliminating a disease state for which administration of a centrally acting non-opioid analgesics, a muscle-relaxant, a functional NMDA antagonist or a substance that increases the expression of the protein Bcl-2 is indicated.

By “pharmaceutically acceptable” it is meant that the carrier, diluent or excipient is compatible with a polymorphic form of flupirtine maleate according to the invention, and not deleterious to a recipient thereof.

The present invention further provides a polymorphic form of flupirtine maleate substantially as hereinbefore described, for use in the manufacture of a medicament for the treatment of a disease state prevented, ameliorated or eliminated by the administration of a centrally acting non-opioid analgesic, a muscle-relaxant, a functional NMDA antagonist and/or an apoptosis inhibitor. More specifically, the present invention provides a polymorphic form of flupirtine maleate, substantially as hereinbefore described, for use in the manufacture of a medicament for treating and preventing a number of disorders including acute and chronic pain, including neuropathic pain, nerve pain, cancer pain, vasomotor and migraine headaches, post-operative pain, post-traumatic pain, burn pain, erosion pain, dysmenorrhoea, dental pain, the pain associated with degenerative and inflammatory joint disease, muscular tension, muscle spasm, muscle stiffness, back pain, neurodegenerative disorders such as Parkinson's disease, dementia including Alzheimer's disease, Huntington's chorea, multiple sclerosis, amyotrophic lateral sclerosis, encephalopathy including AIDS related encephalopathy, Creutzfeldt-Jakob disease including classical and new-variant, Batten disease, diseases of the eye such as maculopathy including senile macular degeneration, diabetic retinopathy, glaucoma, retinitis pigmentosa, myocardial ischaemia and infarction, cerebral ischaemia and infarction, shock, tinnitus and hepatitis.

The present invention also provides a method of treating a disease state prevented, ameliorated or eliminated by the administration of a centrally acting non-opioid analgesic, a muscle-relaxant, a functional NMDA antagonist and/or an apoptosis inhibitor in a patient in need of such treatment, which method comprises administering to the patient a therapeutically effective amount of a polymorphic form of flupirtine maleate, substantially as hereinbefore described. More specifically, the present invention provides a method of treating a number of disorders, including acute and chronic pain, including neuropathic pain, nerve pain, cancer pain, vasomotor and migraine headaches, post-operative pain, post-traumatic pain, burn pain, erosion pain, dysmenorrhoea, dental pain, the pain associated with degenerative and inflammatory joint disease, muscular tension, muscle spasm, muscle stiffness, back pain, neurodegenerative disorders such as Parkinson's disease, dementia including Alzheimer's disease, Huntington's chorea, multiple sclerosis, amyotrophic lateral sclerosis, encephalopathy including AIDS related encephalopathy, Creutzfeldt-Jakob disease including classical and new-variant, Batten disease, diseases of the eye such as maculopathy including senile macular degeneration, diabetic retinopathy, glaucoma, retinitis pigmentosa, myocardial ischaemia and infarction, cerebral ischaemia and infarction, shock, tinnitus and hepatitis, in a patient in need of such treatment, which method comprises administering to the patient a therapeutically effective amount of a polymorphic form of flupirtine maleate, substantially as hereinbefore described.

There is also provided by the present invention a polymorphic form of flupirtine maleate, substantially as hereinbefore described, for use in the manufacture of a medicament for the treatment of a disease state prevented, ameliorated or eliminated by the administration of a centrally acting non-opioid analgesic, a muscle-relaxant, a functional NMDA antagonist and/or an apoptosis inhibitor, wherein the polymorphic form of flupirtine maleate according to the invention, provides an enhanced therapeutic effect compared to the therapeutic effect provided by the existing polymorphic forms of flupirtine maleate. The present invention also provides a corresponding method of treatment, which comprises administering to a patient a therapeutically effective amount of a polymorphic form of flupirtine maleate, substantially as hereinbefore described, so that the administered polymorphic form of flupirtine maleate according to the present invention, provides an enhanced therapeutic effect to the patient, compared to the therapeutic effect provided by corresponding administration of the existing polymorphic forms of flupirtine maleate.

Flupirtine Maleate can be synthesized in accordance with the processes described in the literature, such as U.S. Pat. No. 5,951,15, WO0139760 and WO9505175.

In one embodiment, the present invention provides pharmaceutical compositions comprising at least one of the above flupirtine acid addition salts and their crystalline forms and at least one pharmaceutically acceptable excipient. Optionally, prior known flupirtine salts such as flupirtine maleate and gluconate can also be used in addition to the compounds of the invention.

The pharmaceutical compositions of the invention may also comprise one or more auxiliary excipients such as for example carriers, diluents, binders, lubricants, surfactants, disintegrants, plasticisers, anti-tack agents, opacifying agents, pigments, antioxidants and the like. As will be appreciated by those skilled in the art, the exact choice of excipient and relative amount will depend on the type of pharmaceutical composition, the API, dosage and other factors.

In another embodiment, the present invention also encompasses a pharmaceutical composition comprising at least one of the above described flupirtine acid addition salts and their crystalline forms prepared according to the processes of the present invention, and at least one pharmaceutically acceptable excipient.

In another embodiment, the invention encompasses a process for preparing a pharmaceutical composition comprising combining at least one of the above described flupirtine acid addition salts and their crystalline forms and at least one pharmaceutically acceptable excipient.

In another embodiment, the invention encompasses the use of at least one of the above described flupirtine acid addition salts and their crystalline forms for the manufacture of a medicament for treatment and prevention of acute and chronic pain, pain associated with degenerative and inflammatory joint disease, muscular tension, muscle spasm, muscle stiffness, neurodegenerative disorders, dementia, encephalopathy, diseases of the eye, myocardial ischemia and infarction, cerebral ischemia and infarction, shock, tinnitus and hepatitis.

In another embodiment, the invention encompasses the use of at least one of the above described flupirtine acid addition salts and their crystalline forms for the manufacture of a pharmaceutical composition.

In yet another embodiment, the invention encompasses a method of treating and preventing of acute and chronic pain, pain associated with degenerative and inflammatory joint disease, muscular tension, muscle spasm, muscle stiffness, neurodegenerative disorders, dementia, encephalopathy, diseases of the eye, myocardial ischemia and infarction, cerebral ischemia and infarction, shock, tinnitus and hepatitis, comprising administering a pharmaceutical composition comprising at least one of the above described flupirtine acid addition salts and their crystalline forms to a patient in need thereof.

The flupirtine salts and polymorphs of the present invention are also useful for administration in combination with one or more additional active agent(s). The additional active agent of the present invention can be any compound, agent, molecule, composition, or medication that has biological activity or therapeutic effect. That is, the present flupirtine salts and polymorphs and one or more additional active agent(s) can be co-administered to a patient in need thereof for the treatment of various diseases. The term “co-administration” or “coadministration” refers to administration of the present flupirtine salts and polymorphs and the additional active agent together in a coordinated fashion. The coordinated fashion includes but is not limited to simultaneous, sequential, and separate administration. For example, the co-administration can be simultaneous administration, sequential administration, overlapping administration, interval administration, continuous administration, or a combination thereof. The present flupirtine salts and polymorphs and the at least one additional active agent can be formulated into a single pharmaceutical composition, or can be used in forms of two or more individual pharmaceutical compositions.

The additional active agent includes but is not limited to analgesic medication classes, such as strong and weak opioids, NSAIDs, COX-2 inhibitors, acetaminophen, other anti-inflammatories, tricyclic antidepressants, anticonvulsant agents, voltage gated calcium channel blockers, N-type calcium channel blockers, other calcium channel modulators, SNRIs and other monoamine reuptake inhibitors, sodium channel blockers, NMDA antagonists, AMPA antagonists, other glutamate modulators, GABA modulators, CRMP-2 modulators, NK-1 antagonists, TRPV1 agonists, cannabinoids, adenosine agonists, nicotinic agonists, p38 MAP kinase inhibitors, corticosteroids, and other analgesics.

The present invention can be further illustrated by the following non-limiting Examples.

### Melting Point

Melting point can be measured with the Mettler-Toledo FP apparatus.

### X-Ray Powder Diffraction Analysis (XRPD)

XPRD can be measured with a Philips X'Pert PRO powder diffractometer.

### Differential Scanning Calorimetry (DSC) Analysis

DSC can be measured using a TA Instrument, scanning from 20° C. to 100° C. at a scan rate of 10° C./minute.

### Example I-1

### Preparation of 2-amino-3-carbethoxyamino-6-(4-fluoro-benzylamino)-pyridine acetate

Under nitrogen atmosphere, 20 g of 2-amino-3-carbethoxyamino-6-(4-fluoro-benzylamino)-pyridine is dissolved in 500 ml of Ethanol (90%) at 60° C., 4 g of acetic acid is dissolved in 100 ml of ethanol at 20° C. The mixture is cooled down to 40° C. While stirring the solution of acetic acid is added. The solution is filtered at 20° C. and crystallisation occurred while cooling the solution down to −15° C. for 24 h. The product is collected by filtration and dried at reduced pressure at room temperature for 12 h.

### Example I-2

### Preparation of 2-amino-3-carbethoxyamino-6-(4-fluoro-benzylamino)-pyridine propionate

Under nitrogen atmosphere 10 g of 2-amino-3-carbethoxyamino-6-(4-fluoro-benzylamino)-pyridine and 2.5 g of propionic acid are added to 150 ml of isopropanol. The mixture is heated to 60° C. A transparent solution of flupirtine propionate is formed and is kept for 30 min at 50° C. The solution is filtered at 20° C. and crystallisation occurs while cooling the solution down to −5° C. for 12 h. The product is collected by filtration and dried at reduced pressure at room temperature for 12 h.

### Example I-3

### Preparation of Photomicrographs

The photomicrographs can obtained by gently exerting pressure on the sample material with a mortar and pistil, transferring the sample material to a microscope slide and covering it with immersion oil, followed by observation and image recording on an Optech Biostar microscope equipped with a polarisation filter.

### Example I-4

### Determining the Bulk Density of Flupirtine Salts

The bulk density can be determined as follows: Without increasing its density, sample material is introduced into a 10 ml measuring cylinder (±0.2 ml). The volume and the exact weight of the introduced sample material are determined. The bulk density is calculated as volume per 100.0 g of sample material.

### Example I-5

### Storage Stability of Flupirtine Salts

HPLC can be performed after preparation of the salts and repeated after 14 days of storage at room temperature under normal air containing about 21% oxygen.

### Example I-6

### Dissolution Profiles of Flupirtine Acid Addition Salts in Ethanol

The dissolution in Ethanol can be measured using compacted substance.

Compaction: sample is compacted using a hydraulic press.

Liberation parameter: Basket, 50 UpM, 1000 ml medium 37° C. Apparatus: DT6R Dissolution Tester (ERWEKA).

At given time points 5.0 ml solution is taken and measured vs. reference at 244 nm at a spectral photometer. The solution is refilled with 5 ml solvent. The amount of liberated substance at given time points is calculated using a response factor (absorption/mg substance).

### Example I-7

### Preparation of Flupirtine Triflutate

4 g of trifluoroacetic acid (TFA) is added to a solution of 10 g of 2-amino-3-carbethoxyamino-6-(4-fluoro-benzylamino)-pyridine in 70 ml of isopropanol at 30° C. The solution is stirred for 30 min. Then it is cooled down to −5° C. and kept for 12 h at this temperature. The product can be collected by filtration and dried at reduced pressure at room temperature for 12 h.

### Example II-1

### Preparation of Various Flupirtine Maleate Forms

Flupirtine maleate is dissolved in methyl acetate while heating. Hot solution is filtered and crystallisation occurs while cooling the solution to room temperature. The product is collected by filtration and dried overnight at room temperature.

Flupirtine maleate is placed in a flask filled with a mixture of 96% ethanol and dichloromethane (ratio of 1:2). The reaction mixture is refluxed for 20 minutes while stirring and then cooled in an ice bath to room temperature. Stirring is then continued at room temperature overnight. The product is filtered and dried overnight at room temperature.

Flupirtine maleate is dissolved in 96% ethanol. The solution is heated and the hot solution is filtered and added dropwise to hot methyl acetate. The mixture is stirred at room temperature and precipitation of a crystalline product occurs. The product is filtered.

Flupirtine maleate is placed in a flask filled with a mixture of 96% ethanol and methyl benzoate (ratio of 1:2). The reaction mixture is refluxed for 20 minutes while stirring and then slowly cooled to room temperature. Stirring is continued at room temperature and after 3 hours crystallisation occurs. The reaction mixture is stirred for an additional 3 hours. The product is filtered.

Flupirtine maleate is dissolved in anhydrous methyl benzoate while heating. The resulting solution is placed in a closed bottle to crystallise at room temperature. The product is filtered and dried over night at room temperature.

### Example III-1

### Preparation of 2-amino-3-carbethoxyamino-6-(4-fluoro-benzylamino)-pyridine mesylate

Under nitrogen atmosphere 15 g of 2-amino-3-carbethoxyamino-6-(4-fluoro-benzylamino)-pyridine and 5 g of methane sulfonic acid are added to 500 ml of isopropanol at 20° C. The mixture is heated to 60° C. with stirring for 30 min. A transparent solution is formed. The mixture is cooled down to 5° C. and left overnight for crystallization. Within 12 hours white crystals of flupirtine mesylate are formed. The crystals are removed from the solution by filtration.

### Example III-2

### Preparation of 2-amino-3-carbethoxyamino-6-(4-fluoro-benzylamino)-pyridine besilate

Under nitrogen atmosphere 10 g of 2-amino-3-carbethoxyamino-6-(4-fluoro-benzylamino)-pyridine and 5.3 g of benzene sulfonic acid were added to 330 ml of isopropanol at 20° C. The mixture is heated to 60° C. during stirring for 30 min. A transparent solution is formed. The mixture is cooled down to 5° C. and left overnight for crystallization. The crystals were removed from the solution through filtration.

### Example III-3

### Preparation of 2-amino-3-carbethoxyamino-6-(4-fluoro-benzylamino)-pyridine tosilate

15 g of Flupirtine base were dissolved in 600 ml of Isopropanol at 50° C. 9 g of p-Toluenesulphonic acid were dissolved in 100 ml of Isopropanol at 20° C. At 40° C. the p-Toluenesulphonic acid solution is added to the solution of flupirtine base. Then the mixture is cooled down to 20° C. The mixture is stirred for 60 min. Then the sediment is filtered.

### Example III-6

### Oxidative Stablility of Flupirtine Sulfonic Acid Salts

HPLC analysis can be performed after isolation and drying of flupirtine sulfonate salts and repeated after 14 days of storage at room temperature under open air.

### Example III-6

### Bulk Density of Flupirtine Sulfonate Salts

The bulk density can be determined as follows: Without increasing its density, sample material is introduced into a 10 ml measuring cylinder (±0.2 ml). The volume and the exact weight of the introduced sample material are determined. The bulk density is calculated as volume per 100.0 g of sample material.

### Example III-7

### Solubility of Flupirtine Sulfonate Salts

The solubility can be determined by placing 500 mg of sample material in a 250 ml glass beaker, adding 100 ml of demineralised water and agitating the suspension for 2 hours on a shaker (300 rpm). Subsequently, an aliquot of the solution is centrifuged at 10,000 rpm and the flupirtine absorption is photometrically determined in the supernatant. The amount of flupirtine in solution is calculated with a response factor (absorption/mg substance).

### Example III-8

### Discolouration Measurements

Discolouration can be measured using optical comparison with a standard: the RAL scale of colours (see list). The colour changes during the experiment is compared to this standard list and the appropriate value is noted. The intensity is increasing on a scale ranging from 1 to 10. A higher number means more discolouration. The increase in colour intensity is not proportional but qualitative.

### Example IV-1

### Rat Intestinal Perfusion Test

The rat intestine perfusion technique is a direct way to measure the regional absorption properties of a test compound in the gastrointestinal tract. Rat intestinal permeability coefficient (Peff) can be used to predict human in vivo oral absorption of passively absorbed compounds. Fagerholm, M. Johansson, and H. Lennernas, “Comparison between permeability coefficients in rat and human jejunum”, Pharm. Res., 13, 1996, 1336 1342, have demonstrated a good correlation between rat Peff and human fraction of dose absorbed (Fa) for a series of compounds. Other characteristics such as formulable Maximum Absorbable Dose (MAD), FDA Biopharmaceutical Classification, etc. can also be estimated using this test and will provide important information on the compound.

The perfusions will be performed in three intestinal sections of anesthetized rats: duodenum-jejunum, ileum, and colon. The lengths of the segments were approximately 10 12 cm for small intestine segments and 5 6 cm for colon segments. An inflow cannula will be inserted at the proximal end and an outflow cannula was inserted at the distal end. Perfusate was pumped through the segment at 0.19 mL/min, and collected at 20, 40, 55, 70, 85 and 100 minutes.

Selected salt(s) or/and polymophs of flupirtine will be added for testing to the perfusion in concentration equivalent to the target human dose. The disappearance rates of flupirtine compound, metoprolol, and glucose will be determined from each collection interval by comparing to the initial compound solution remaining in the syringe at the end of the 100 minutes. This is to correct for any losses due to binding to the syringe or tubing. Meanwhile, drug concentration in perfusate samples will be corrected for water influx/efflux. Effective intestinal permeability, Peff (cm/sec), will be determined.

In general, the results will show the absorption rates for the selected drugs and will allow to select the most optimal form for increase absorption. The solubility of water at room temperature will also be considered in the final selection.

Studies on microdosing might be considered to determine an optimize dose range and explore potential for QD formulation and other characteristics of the drug.

All publications and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A crystalline salt of flupirtine, which is a carboxylic acid salt or a sulfonic acid salt.

2. The crystalline salt of claim 1, wherein the carboxylic acid has a structural formula selected from the following: 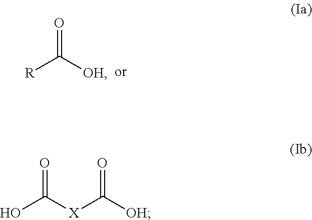
wherein R is a C1 to C20 alkyl, C6 to C10 aryl, or a C2 to C20 alkenyl; and X is a C1 to C6 alkylene or a C2 to C6 alkenylene.

3. The crystalline salt of claim 1, wherein the sulfonic acid has a structural formula (II): 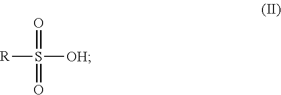
wherein R is a C1 to C20 alkyl, C6 to C10 aryl, or a C2 to C20 alkenyl.

4. The crystalline salt of claim 2, which is a salt of a carboxylic acid having structural formula (Ia), wherein R represents hydrogen or a C₁-C₆ alkyl group.

5. The crystalline salt of claim 4, wherein R is a saturated C₁-C₆ alkyl group, with the proviso that RCOOH is not &conic acid.

6. The crystalline salt of claim 5, wherein R is selected from a group consisting of: hydrogen, methyl, ethyl, n-propyl, iso-propyl, n-butyl, sec-butyl, tert-butyl, pentyl and hexyl.

7. The crystalline salt of claim 4, which is formic, acetic or propionic acid addition salt.

8. The crystalline salt of claim 4, which has a non-acicular morphology.

9. The crystalline salt of claim 4, which is stable towards oxidation and/or discoloration when exposed to air at room temperature for about 30 days.

10. The crystalline salt of claim 4, having a solubility of at least 1% by weight in an alcohol-containing solution having an alcohol content of at least 10% by volume of the solution.

11. The crystalline salt of claim 4, wherein the mole ratio of the acid component to the flupirtine base component is 1:1 to 1:1.3, respectively.

12. A pharmaceutical composition comprising the carboxylate acid addition salt of flupirtine of claim 4, and at least one pharmaceutically acceptable excipient.

13. The crystalline salt of claim 2, which is a polymorph of flupirtine maleate.

14. A pharmaceutical composition comprising a therapeutically effective dose of a polymorphic form of flupirtine maleate of claim 13; and a pharmaceutically acceptable excipient.

15. The crystalline salt of claim 3, which is a salt of a sulfonic, acid having structural formula (II), wherein R is a C₁-C₁₂ alkyl or C₆-C₁₀ aryl group.

16. The crystalline salt of claim 15, wherein the acid component is selected from the list consisting of methane sulfonic acid, ethane sulfonic acid, n-propane sulfonic acid, n-butane sulfonic acid, dodecane sulfonic acid, phenylethane sulfonic acid, hydroxyethane sulfonic acid, methoxyethane sulfonic acid, benzene sulfonic acid, halogenbenzene sulfonic acid, methoxybenzene sulfonic acid, toluene sulfonic acid, 5-salicylsulfonic acid, naphthalene sulfonic acid, and naphthalene disulfonic acid.

17. The crystalline salt of claim 16, wherein the halogenbenzene sulfonic acid is selected from the list consisting of 4-fluorobenzene sulfonic acid, 4-chlorobenzene sulfonic acid and 4-bromobenzene sulfonic acid.

18. The crystalline salt of claim 16, wherein the naphthaline sulfonic acid is selected from the list consisting of 1-naphthaline sulfonic acid and 2-naphthaline sulfonic acid.

19. The crystalline salt of claim 16, wherein the acid is methane sulfonic acid or benzene sulfonic acid.

20. The crystalline salt of claim 15, wherein R represents an substituted methyl, ethyl, n-propyl, isopropyl, n-butyl, sec-butyl, tert-butyl, pentyl, hexyl, cyclohexyl, heptyl, octyl, nonyl, decyl, dodecyl, phenyl or naphthyl moiety.

21. The crystalline salt of claim 15, wherein R is substituted with hydroxyl, C₁-C₄-alkoxy, phenoxy, halogen, COOH, C₁-C₄-alkyl, or phenyl.

22. The crystalline salt of claim 15, wherein the crystalline salt has a short column structure.

23. The crystalline salt of claim 15, wherein the salt is stable towards oxidation and/or discoloration when exposed to air at about room temperature for about 30 days.

24. The crystalline salt of claim 15, wherein the molar ratio of flupirtine to acid is 1:1.3, respectively.

25. A pharmaceutical composition comprising the sulfonate addition salt of flupirtine of any of the claim 15, and at least one pharmaceutically acceptable excipient.

26. A crystalline salt of flupirtine according to claim 1 for use in therapy.
